(19) United States
(12) Patent Application Publication
(10) Pub. No.: US 2021/0251316 A1
(43) Pub. Date: Aug. 19, 2021

(54) PROTECTIVE SLEEVE FOR FINGERS AND TOES

(71) Applicant: BELRAGONA LLC, Chicago, IL (US)

(72) Inventors: Joseph David Belmonte, Bensenville, IL (US); Rosalba Ragona, Lombard, IL (US)

(73) Assignee: BELRAGONA LLC, CHICAGO, IL (US)

(21) Appl. No.: 17/174,467

(22) Filed: Feb. 12, 2021

Related U.S. Application Data
(60) Provisional application No. 62/976,074, filed on Feb. 13, 2020.

(51) Int. Cl. A41D 13/08; A41D 13/06; A61M 35/00; A61K 31/05; A61K 47/14

(52) U.S. Cl. CPC A41D 13/087; A41D 13/06; A61M 35/10; A61M 2210/083; A61K 47/14; A41D 2400/32; A61M 2210/086; A61K 31/05

(57) ABSTRACT

A digit protection assembly includes an outer shell that includes a moisture repellant material. A strap is operably coupled to the outer shell. The strap includes a fastener that is operably coupled to an attachment end of the strap. An inner liner is operably coupled to the outer shell and defines a pocket between the inner liner and the outer shell. The inner liner includes an absorbent material. A medication is disposed on the inner liner. The medication comprises cannabidiol oil.

### CROSS REFERENCE TO RELATED APPLICATION

This application claims the benefit under 35 U.S.C. § 119(e) to U.S. Provisional Patent Application No. 62/976,074, filed on Feb. 13, 2020, entitled “PROTECTIVE SLEEVE FOR FINGERS AND TOES,” which is incorporated herein by reference in its entirety.

### FIELD OF THE DISCLOSURE

The present disclosure generally relates to protective sleeves, and more particularly to protective sleeves for protecting fingers and toes.

### SUMMARY OF THE DISCLOSURE

According to one aspect of the present disclosure, a protective sleeve for a digit includes an outer shell that defines slits and has a tapered configuration. Straps extend outwardly from and are operably coupled to the outer shell. The straps each include an attachment end and a stabilizing portion that is coupled to the outer shell. A fastener is operably coupled to the attachment end and is selectively coupled to at least one of the outer shell and the stabilizing portions of the straps. An inner liner is operably coupled to the outer shell to define a pocket between the inner liner and the outer shell. The inner liner includes a plurality of layers and includes an absorbent material. A rigid member is disposed within the pocket via one of the slits defined by the outer shell. An oil is disposed on at least one layer of the plurality of layers of the inner liner.

According to another aspect of the present disclosure, a digit protection assembly includes an outer shell that includes a moisture repellant material. A strap is operably coupled to the outer shell. The strap includes a fastener that is operably coupled to an attachment end of the strap. An inner liner is operably coupled to the outer shell and defines a pocket between the inner liner and the outer shell. The inner liner includes an absorbent material. A medication is disposed on the inner liner. The medication comprises a cannabidiol oil.

According to yet another aspect of the present disclosure, a protective sleeve assembly includes an outer shell that has an inner surface and a fastening material that is disposed on the inner surface. A strap extends outwardly from the outer shell. The strap has an attachment end that is selectively coupled to the outer shell. A fastener is operably coupled to the attachment end of the strap. The fastener includes a first coupling feature that is selectively coupled to at least one of the outer shell and the strap. An inner liner includes a first layer and a second layer, and the first layer is operably coupled to the inner surface of the outer shell. A cannabidiol oil is disposed on the second layer of the inner liner.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a top perspective view of a hand with a protective sleeve of the present disclosure applied to a digit of a user;

FIG. 2 is a top perspective view of a hand with a protective sleeve of the present disclosure applied to a digit and an adjacent digit of a user;

FIG. 3 is a bottom perspective view of a protective sleeve of the present disclosure;

FIG. 4 is an exploded bottom perspective view of the protective sleeve of FIG. 3 with an outer shell and an inner liner;

FIG. 5 is a top perspective view of a protective sleeve of the present disclosure with an outer shell defining slits and straps;

FIG. 6 is a partial enlarged top perspective view of the outer shell of FIG. 5 and the slits defined by the outer shell taken at area VI;

FIG. 7 is a cross-sectional view of the protective sleeve of FIG. 6 taken along lines VII-VII;

FIG. 8 is an exploded top perspective view of an inner liner of a protective sleeve of the present disclosure; and

FIG. 9 is an enlarged top perspective view of a medication layer of the inner liner of FIG. 8.

The components in the figures are not necessarily to scale, emphasis instead being placed upon illustrating the principles described herein.

### DETAILED DESCRIPTION OF THE EMBODIMENTS

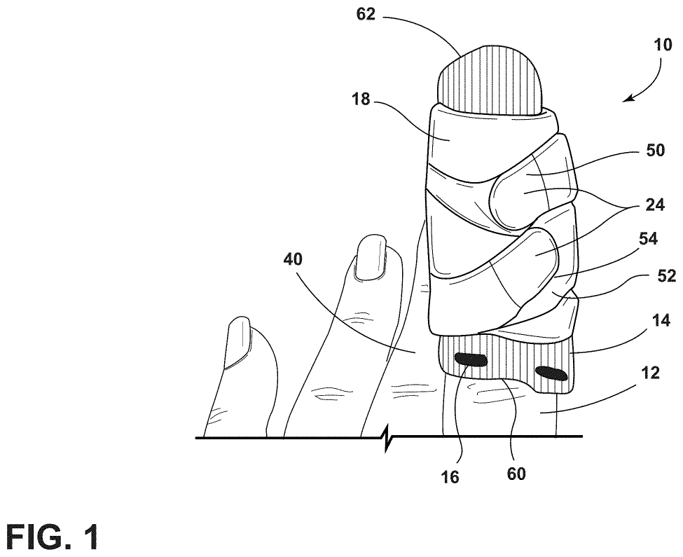

For purposes of description herein, the terms “upper,” “lower,” “right,” “left,” “rear,” “front,” “vertical,” “horizontal,” and derivatives thereof shall relate to the disclosure as oriented in FIG. 1A. However, it is to be understood that the disclosure may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

In this document, relational terms, such as first and second, top and bottom, and the like, are used solely to distinguish one entity or action from another entity or action, without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

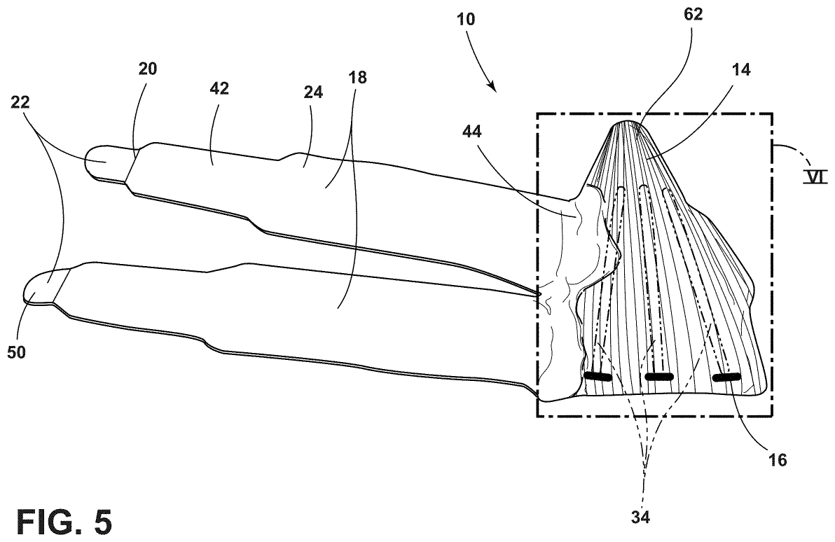

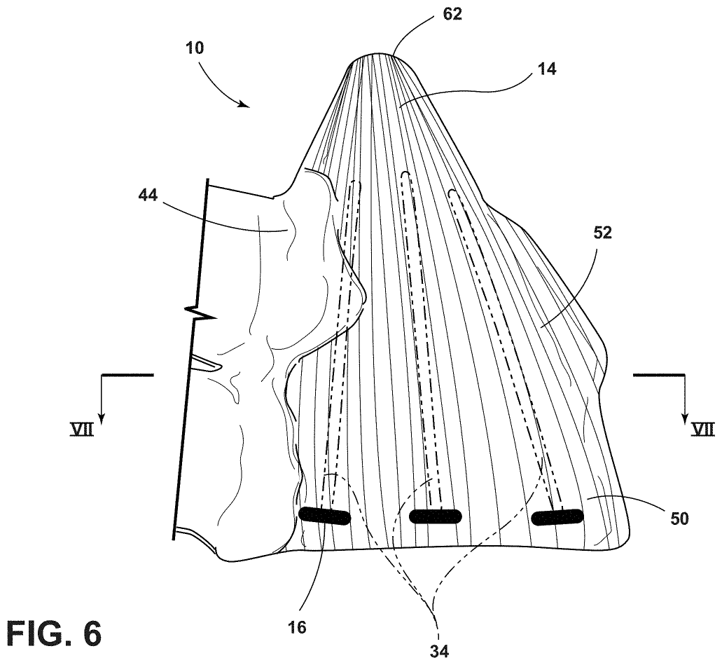

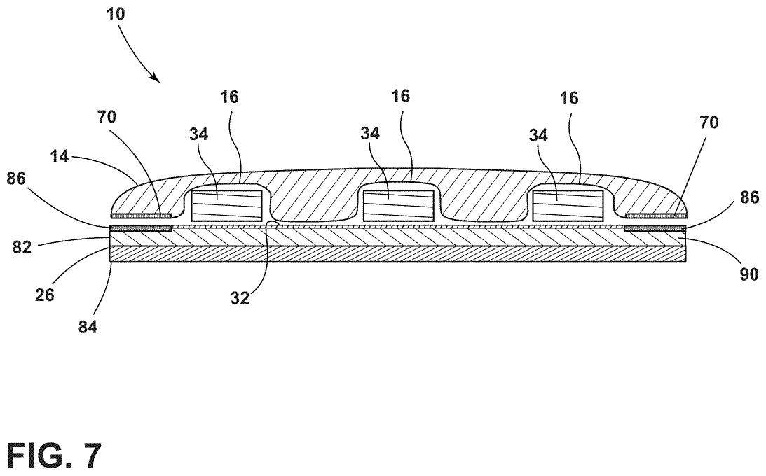

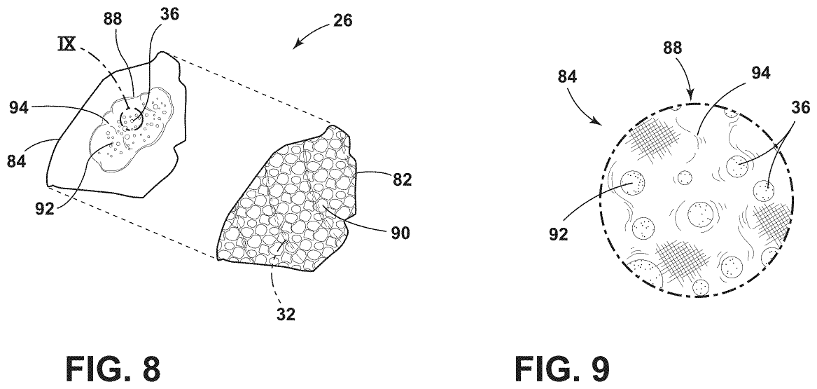

Referring to FIGS. 1-9, reference numeral 10 generally designates a protective sleeve for a digit 12. The protective sleeve 10 includes an outer shell 14 that can define slits 16 and can have a tapered configuration. Straps 18 extend outwardly from and are operably coupled to the outer shell 14. Each strap 18 includes an attachment end 20 and a stabilizing portion 22 is coupled to the outer shell 14. A fastener 24 is operably coupled to the attachment end 20 and is selectively coupled to at least one of the outer shell 14 and the stabilizing portions 22 of the straps 18. An inner liner 26 is operably coupled to the outer shell 14 to define a pocket 28 between the inner liner 26 and the outer shell 14. The inner liner 26 includes a plurality of layers 30 and includes an absorbent material 32. A rigid member 34 is disposed within the pocket 28 via one of the slits 16 defined by the outer shell 14, and a oil 36 is disposed on at least one layer of the plurality of layers 30 of the inner liner 26.

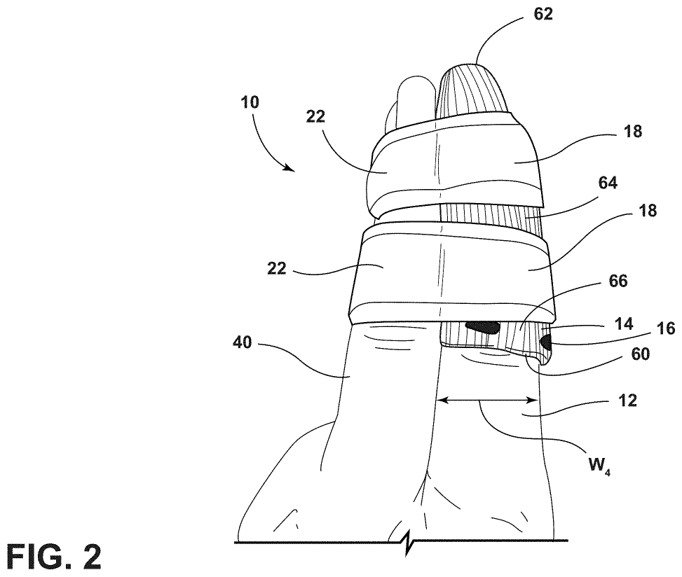

Referring to FIGS. 1-3, the protective sleeve 10 may be referred to as a protective sleeve assembly and/or a digit protection assembly, which includes the outer shell 14, the straps 18, and the inner liner 26. The protective sleeve 10 may generally be conical in shape, such that the protective sleeve 10 is illustrated with the tapered configuration. Additionally or alternatively, the protective sleeve 10 may include alternate shapes and configurations depending on the size and length of the digit 12. The protective sleeve 10 may have a variety of shapes and sizes, such that the protective sleeve 10 may be used with any one of a user's digits. While the digit 12 illustrated is a user's finger, it is also contemplated that the protective sleeve 10 may be used with other digits, such as toes. Accordingly, the term digit 12 herein may refer to either fingers or toes. As will be described in more detail below, the protective sleeve 10 is ultimately secured to the digit 12 via the straps 18. The straps 18 can extend around the digit 12 on which the protective sleeve 10 is positioned to secure the protective sleeve 10 to the digit 12, as illustrated in FIG. 1. Additionally or alternatively, the straps 18 can extend around an adjacent digit 40 to securely couple the digit 12 and the protective sleeve 10 with the adjacent digit 40, as illustrated in FIG. 2. In this alternate configuration, the adjacent digit 40 may provide additional support for the digit 12 within the protective sleeve 10, as the digit 12 within the protective sleeve 10 may be injured or otherwise need additional bracing.

As mentioned above, the straps 18 extend outwardly from and are operably coupled to the outer shell 14. Additionally or alternatively, the straps 18 may be integrally formed with the outer shell 14, such that the straps 18 may be formed from the same material as the outer shell 14, described in more detail below. The stabilizing portion 22 of each of the straps 18 is coupled to the outer shell 14. Each of the straps 18 may also include an attachment portion 42 disposed between the attachment end 20 and the stabilizing portion 22. The attachment portion 42 is generally elastic or flexible, such that the attachment portion 42 may be referred to as an elastic portion. The attachment portion 42 can flexibly extend and retract to assist the user in securing the straps 18 about the digit 12. It is generally contemplated that the stabilizing portion 22 can have a width W₁ that is greater than a width W2 of the attachment portion 42. Additionally or alternatively, the width W2 of the attachment portion 42 may be greater than the width W₁ of the stabilizing portion 22. Further, the width W2 of the attachment portion 42 and the width W₁ of the stabilizing portion 22 may be generally equal or otherwise uniform.

With further reference to FIGS. 1-3, the stabilizing portion 22 of each strap 18 may be generally inelastic to provide additional support for the digit 12 within the protective sleeve 10. The inelasticity of the stabilizing portion 22 advantageously provides stability and added support to the digit 12 when the stabilizing portion 22 is disposed around the protective sleeve 10. The stabilizing portion 22 is relatively flexible, while inelastic, to bend and wrap around the digit 12 to secure the protective sleeve 10. Additionally or alternatively, the stabilizing portion 22 may be configured to be partially elastic to minimize potential strain placed on the digit 12 while the protective sleeve 10 is secured.

The attachment portion 42 of each of the straps 18 may be generally elastic or flexible, such that the attachment portion 42 may flex and extend around the digit 12 while the protective sleeve 10 is secured around the digit 12. The attachment end 20, mentioned above, is defined as the end of the attachment portion 42 to which the fastener 24 is coupled. The attachment end 20 of the straps 18 is defined as a distal end of the straps 18 relative to the outer shell 14 and the inner liner 26, such that a proximal end 44 of the straps 18 is coupled to the outer shell 14. The fasteners 24 are coupled to the attachment end 20, as mentioned above, and may include a coupling feature 46 disposed on a first side 48 of each of the fasteners 24. A second side 50 of the fasteners 24 may be formed from a grip material, such as silicon, to assist the user in attaching the fastener 24 to secure the protective sleeve 10. The grip material of the second side 50 may minimize slipping or general loss of grip during adjustment or attachment of the fastener 24. The second side 50 may also be formed from other practicable materials generally known in the art.

Referring still to FIGS. 1-3, it is generally contemplated that the coupling feature 46 of each fastener 24 may be a first coupling feature. Stated differently, the first coupling feature 46 may be disposed on the first side 48 of each of the fasteners 24, and a second coupling feature 52 may be disposed on the outer shell 14. The second coupling feature 52 may be a reciprocal coupling feature to the first coupling feature 46. The first coupling feature 46 is configured to couple to the second coupling feature 52, such that the first coupling feature 46 and the second coupling feature 52 can be releasably coupled to one another. It is contemplated that a hook and loop fastening system may be used for the first and second coupling features 46, 52, or other like coupling systems. The first coupling feature 46, and the second coupling feature 52 may be repeatedly coupled and uncoupled. The straps 18 are configured to wrap around at least the digit 12 and the protective sleeve 10 to secure the protective sleeve 10 around the digit 12, as mentioned above. The straps 18 may be wrapped around the digit 12 and/or the adjacent digit 40, such that portions of the stabilizing portion 22 may overlap. The stabilizing portion 22 may provide additional bracing or support for the digit 12 within the protective sleeve 10 as the stabilizing portion 22 is generally inelastic.

The attachment portion 42 may ultimately provide the elastic attachment and flexibility of each of the straps 18 to secure the protective sleeve 10 on the digit 12. As mentioned above, the second coupling feature 52 may be disposed on and/or integrally formed with the outer shell 14. It is also contemplated that the second coupling feature 52 may be integrally formed and/or disposed on a fastening surface 54 of the straps 18. For example, the straps 18 may wrap around the digit 12 and the protective sleeve 10, and the first coupling feature 46 of each fastener 24 may operably couple to the second coupling feature 52 disposed on the fastening surface 54 of the straps 18. Stated differently, the fastener 24 of each of the straps 18 may operably couple to either the fastening surface 54 of either strap 18 and/or the second coupling feature 52 disposed on the outer shell 14.

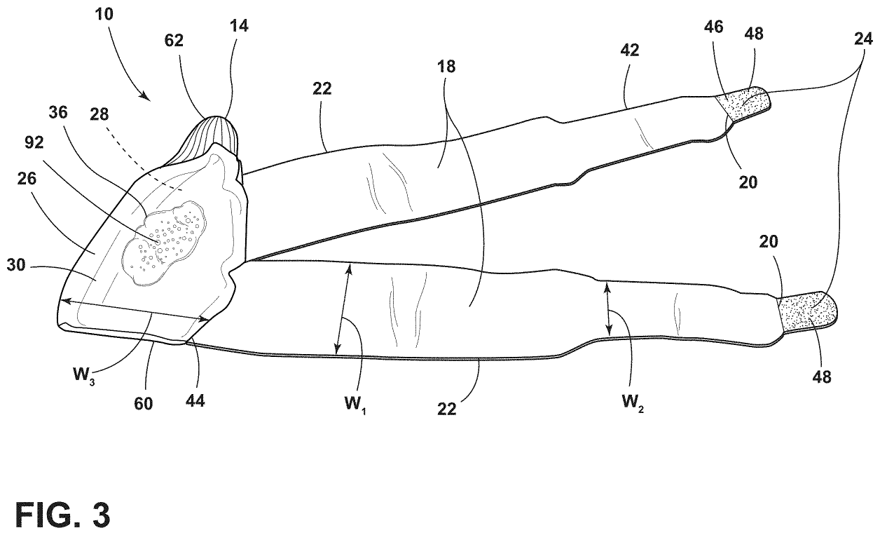

With reference now to FIGS. 2-4, the protective sleeve 10 includes a receiving end 60, a bounded end 62, and a body portion 64 therebetween. As illustrated in FIG. 3, the receiving end 60 generally tapers toward the bounded end 62. It is generally contemplated that the receiving end 60 is open and/or flat to assist in placement of the digit 12. The receiving end 60 of the protective sleeve 10 may have a width W3 that is generally twice that of a width W4 of the digit 12 on which the protective sleeve 10 is to be positioned. By way of example, not limitation, the width W4 of the digit 12 may be approximately 2-millimeters and the selected protective sleeve 10 may have a width W3 of approximately 4-millimeters. The width W3 of the protective sleeve 10 may be greater than 4-millimeters or less than 4-millimeters depending on the size of the digit 12. Stated differently, the width W3 of the protective sleeve 10 may be less than, equal to, or greater than the width W4 of the digit 12.

The bounded end 62 is illustrated as being at least partially enclosed, such that a portion of the digit 12 may be at least partially positioned within the bounded end 62. Additionally or alternatively, the bounded end 62 may be closed, such that the digit 12 may abut the bounded end 62. In this alternate configuration, the bounded end 62 may provide additional padding for the digit 12 and may partially brace the digit 12 within the protective sleeve 10. If the digit 12 is positioned within the bounded end 62, then the bounded end 62 may assist the user in placing the protective sleeve 10 on the digit 12. For example, the bounded end 62 can at least partially retain the protective sleeve 10 on the digit 12 prior to the user securing the protective sleeve 10 with the straps 18, as described above.

Referring still to FIGS. 2-4, the body portion 64 of the protective sleeve 10 includes the outer shell 14 and the inner liner 26. The outer shell 14 can be formed from a generally rigid material configured to provide support for the digit 12, while remaining sufficiently flexible to define a supportive fit around the digit 12. Stated differently, the outer shell 14 can be generally pliably rigid, such that the protective sleeve 10 can be molded or otherwise shaped around the digit 12. It is also contemplated that the outer shell 14 may be at least partially elastically deformable and may be configured to at least partially provide a compressive force on the digit 12. Typically, the protective sleeve 10 is used with an injured digit 12, such that the protective sleeve 10 is assisting in a healing process of the digit 12. Thus, it can be advantageous for the outer shell 14 of the protective sleeve 10 to provide additional compression on the digit 12, depending on the injury.

It may also be advantageous for the outer shell 14 to be generally pliably rigid, such that the outer shell 14 may provide at least partial protection of the digit 12 while still allowing for minimal movement of the digit 12. The outer shell 14 may be formed from materials including, but not limited to, nylon, spandex, an elastomeric material, fine-knit interlocking fibers, and other practicable materials. It is also contemplated that the outer shell 14 may be formed from a moisture wicking and/or moisture repellant material, such that the outer shell 14 is generally water resistant and/or repellant.

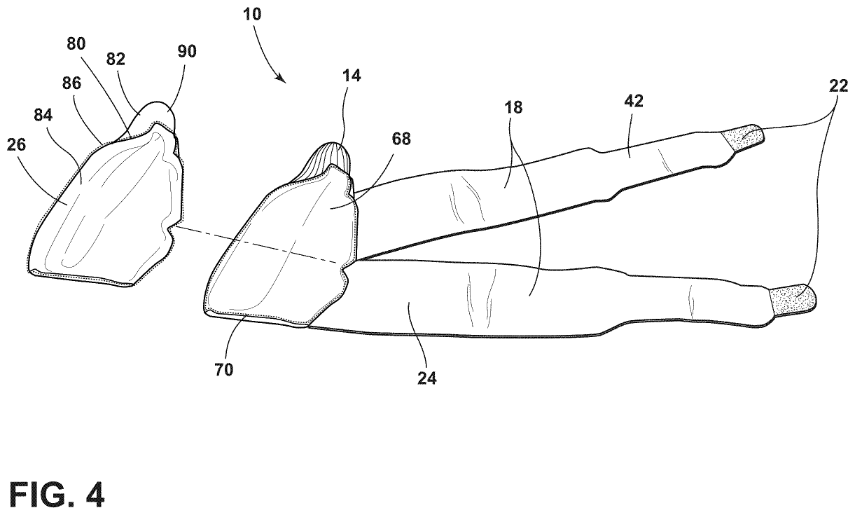

As mentioned above, the outer shell 14 can define slits 16 along a lower edge 66 of the outer shell 14, and the pocket 28 is defined between the outer shell 14 and the inner liner 26. The slits 16 may generally provide access to the pocket 28, as described below. As illustrated in FIG. 4, the outer shell 14 includes an inner surface 68 on which a fastening material 70 is disposed. The inner liner 26 is operably and removably coupled to the inner surface 68 of the outer shell 14 via the fastening material 70, as described further below. The fastening material 70 may be a hook material similar to the first coupling feature 46, described above with respect to the straps 18. It is also contemplated that the fastening material 70 may be other materials generally known for fastening. The inner liner 26 is configured to couple to the fastening material 70 to retain the inner liner 26 with the outer shell 14, described in more detail below.

With reference to FIGS. 2 and 4-7, the rigid member 34, mentioned above, may be positioned within the pocket 28 defined between the outer shell 14 and the inner liner 26. For example, the rigid member 34 may be inserted into at least one of the slits 16 defined along the lower edge 66 of the outer shell 14. While the slits 16 are illustrated along the lower edge 66 of the outer shell 14, it is also contemplated that the slits 16 may be defined at any practicable location along the outer shell 14. The rigid member 34 may include multiple rigid members 34 depending on the desired rigidity of the protective sleeve 10. By way of example, not limitation, the rigid member 34 may be a splint configured to brace the digit 12. The rigid member 34 is configured to assist in maintaining the digit 12 in a predetermined position. For example, the rigid member 34 may be configured to assist the protective sleeve 10 in maintaining the digit 12 in a straight position relative to the adjacent digit 40. The straps 18, described above, can also assist in maintaining the predetermined position of the digit 12 in combination with the rigid members 34.

Referring now to FIGS. 2 and 7-9 and as mentioned above, the protective sleeve 10 also includes the inner liner 26, which is operably coupled to the outer shell 14. A peripheral edge 80 of the inner liner 26 is operably coupled to the outer shell 14 to define the pocket 28 between the inner liner 26 and the outer shell 14. The pocket 28 may be accessed via the slits 16 defined by the outer shell 14, such that the rigid members 34 may be disposed within the pocket 28. The inner liner 26 also includes the plurality of layers 30, which may include at least a first layer 82 and a second layer 84. The inner liner 26 is operably coupled to the outer shell 14 along the peripheral edge 80 of the inner liner 26. The peripheral edge 80 of the inner liner 26 can be formed from a second, reciprocal fastening material 86 that may be retained by the fastening material 70 (FIG. 4) disposed on the inner surface 68 of the outer shell 14. Stated differently, the second fastening material 86 is reciprocally coupled to the first fastening material 70 to couple the inner liner 26 to the outer shell 14. As mentioned above with respect to the first and second coupling features 46, 52 of the straps 18, it is contemplated that a hook and loop fastening system may be used for the first and second fastening materials 70, 86 (FIG. 4), or other like coupling systems.

The inner liner 26 may be repeatedly coupled and uncoupled from the outer shell 14 via the second fastening material 86. Stated differently, the inner liner 26 is removably coupled to the outer shell 14. It is also contemplated that the inner liner 26 may be repeatedly removed and replaced with a new inner liner 26, such that the inner liner 26 is configured to be disposable and/or replaceable. Thus, the user may advantageously repeatedly use the protective sleeve 10 while minimizing potential exposure that may occur from reusing a medical device. Specifically, the inner liner 26 may be configured to receive medication 88, such as the oil 36, as described further below. The inner liner 26 may be formed from gauze, silicone, elastomeric materials, rubber latex, and other practicable materials.

With further reference to FIGS. 2 and 7-9, the first layer 82 of the inner liner 26 may include a phase change material 90 either disposed on and/or integrated with the first layer 82. The phase change material 90 may include, but is not limited to, a gel pad, microfiber cooling gel beads, a beaded cooling aid, ammonium nitrate or urea, magnesium sulfate, calcium chloride, and/or a soft shock absorbing gel. The second layer 84 is configured to receive the oil 36 and will be described in more detail below. The phase change material 90 may comprise the first layer 82, such that the first layer 82 may be referred to as the phase change material 90. The phase change material 90 may take the form of a gel or other malleable semisolid and is configured to facilitate thermal transfer between the inner liner 26 and the digit 12. It is also contemplated that the first layer 82 may be formed from a rubber latex and/or a silicone material in which the phase change material 90 may be disposed. By way of example, and not limitation, the protective sleeve 10 may be positioned within a cooling compartment, such as a freezer, to generally transition the phase change material 90 into a solid or semi-solid state. It is also contemplated that the phase change material 90 may remain pliable or otherwise malleable in the solid-state, such that the inner liner 26 may be molded or otherwise positioned around the digit 12.

It is also contemplated that the protective sleeve 10 may be warmed or otherwise heated in order to thermally transfer heat to the digit 12 from the phase change material 90. The phase change material 90 may be disposed proximate to the outer shell 14, such that the phase change material 90 at least partially defines the pocket 28 between the inner liner 26 and the outer shell 14. The rigid member 34 positioned within the pocket 28 is generally thermally resistant, such that the heating or cooling of the phase change material 90 generally does not affect the rigid member 34. It is also contemplated that the rigid member 34 may be removed during the heating or cooling process of the phase change material 90. It is further contemplated that the phase change material 90 may include chemicals configured to undergo an endothermic or exothermic process that independently heats or cools the first layer 82.

As briefly mentioned above, the second layer 84 may receive the oil 36 or other medication 88. The second layer 84 may be formed from a padded material that is at least partially absorbent. By way of example, not limitation, the added material of the second layer 84 may be gauze, cotton, or any other practicable material configured to at least partially absorb the oil 36. The second layer 84 is positioned proximate to the digit 12, such that the digit 12 contacts the second layer 84 of the inner liner 26. It is generally contemplated that the second layer 84 may be referred to as a medication layer, such that the medication 88, which may include the oil 36, may be disposed on the medication layer 84. It is also contemplated that the medication 88 may take the form of a semisolid or ointment, such as a gel. For example, the medication 88 may include, but is not limited to, anti-bacterial medication, burn relief ointment, anti-inflammatory medication, comfort gels, cooling gels, arthritic medication, healing oils, vitamin E, and/or cannabidiol oil.

With further reference to FIGS. 2 and 7-9, the medication layer 84 may include the absorbent material 32. It is generally contemplated that the absorbent material 32 may include either a water absorbent material or an oil absorbent material. The absorbent material 32 is configured to generally retain the medication 88 on the medication layer 84 prior to insertion of the digit 12. The medication 88 can be at least partially transferred to the digit 12 from the medication layer 84 when the protective sleeve 10 is positioned on the digit 12. One of the medications 88 that can be applied to the medication layer 84 is a cannabidiol (CBD) oil 92, mentioned above. The CBD oil 92 is one of the identified cannabinoids in cannabis plants and may account for up to approximately 40% of the extract from the cannabis plants. It is generally contemplated that the CBD oil 92 may react with the user's neuroreceptors to assist in pain management. CBD oil 92 can be extracted from the cannabis plants and can be combined with an emulsifier and/or a dispersant to evenly apply the CBD oil 92 to the medication layer 84.

The CBD oil 92 may include full-spectrum CBD, broad-spectrum CBD, and or CBD isolate. The CBD oil 92 is typically applied to the medication layer 84 via a carrier oil 94. The carrier oil 94 may include, but is not limited to, coconut oil, avocado oil, jojoba oil, apricot kernel oil, sweet almond oil, olive oil, organ oil, rose hip oil, flaxseed oil, grape seed oil, sunflower oil, macadamia nut oil, vegetable glycerin, and/or a medium chain triglyceride oil. It is advantageous to combine approximately 1% to 20% of the CBD oil 92 with the carrier oil 94 on the medication layer 84. It is also contemplated that less than 1% or greater than 20% of the CBD oil 92 may be combined with the carrier oil 94 prior to application to the medication layer 84. It is also contemplated that the CBD oil 92 may be water-soluble, such that the digit 12 may be readily cleaned after use of the protective sleeve 10. While the CBD oil 92 is combined with the carrier oil 94 to form the oil 36 mentioned above, it is also contemplated that the oil 36 may be combined with any one of the medications 88 mentioned above. It is further contemplated that the oil 36 may be the medication 88 that is applied to the medication layer 84 independent of any additional medication 88.

Referring again to FIGS. 1-9, the protective sleeve 10 along with the CBD oil 92 on the medication layer 84 is configured to assist in alleviating potential pain of the digit 12. The user may apply the CBD oil 92, along with the carrier oil 94, to the medication layer 84 for direct contact between the CBD oil 92 and the digit 12. The user may also remove and replace the inner liner 26, including the medication layer 84, to maintain the overall cleanliness of the protective sleeve 10. The user may thus reuse the protective sleeve 10 repeatedly by removing and replacing the inner liner 26. The fastening material 70 on the outer shell 14 assists in the selective retention of the inner liner 26. The outer shell 14 can also define the slits 16 through which the rigid members 34 may be positioned.

The invention disclosed herein is further summarized in the following paragraphs and is further characterized by combinations of any and all of the various aspects described therein.

According to one aspect of the present disclosure, a protective sleeve for a digit includes an outer shell that defines slits and has a tapered configuration. Straps extend outwardly from and are operably coupled to the outer shell. The straps each include an attachment end and a stabilizing portion that is coupled to the outer shell. A fastener is operably coupled to the attachment end and is selectively coupled to at least one of the outer shell and the stabilizing portions of the straps. An inner liner is operably coupled to the outer shell to define a pocket between the inner liner and the outer shell. The inner liner includes a plurality of layers and includes an absorbent material. A rigid member is disposed within the pocket via one of the slits defined by the outer shell. An oil is disposed on at least one layer of the plurality of layers of the inner liner.

According to another aspect, an oil includes a cannabidiol oil.

According to another aspect, an oil includes a cannabidiol oil and a carrier oil.

According to another aspect, straps further include an elastic portion that are disposed between an attachment end and a stabilizing portion.

According to another aspect, at least one layer of a plurality of layers includes a medication layer. An absorbent material is disposed over the medication layer.

According to another aspect, at least one layer of a plurality of layers includes a phase change material.

According to another aspect of the present disclosure, a digit protection assembly includes an outer shell that includes a moisture repellant material. A strap is operably coupled to the outer shell. The strap includes a fastener that is operably coupled to an attachment end of the strap. An inner liner is operably coupled to the outer shell and defines a pocket between the inner liner and the outer shell. The inner liner includes an absorbent material. A medication is disposed on the inner liner. The medication comprises cannabidiol oil.

According to another aspect, a cannabidiol oil comprises 20% of a medication.

According to another aspect, an inner liner includes a first layer and a second layer. A first layer includes a phase change material.

According to another aspect, a second layer includes an absorbent material and a medication.

According to another aspect, an outer shell has an inner surface and a fastening material that is disposed on the inner surface. An inner liner includes a second fastening material that is reciprocally coupled to the fastening material of the outer shell.

According to another aspect, an outer shell defines a slit along a lower edge of the outer shell.

According to another aspect, a digit protection assembly further includes a rigid member that extends through a slit defined by an outer shell and is disposed within a pocket defined between an inner liner and the outer shell.

According to yet another aspect of the present disclosure, a protective sleeve assembly includes an outer shell that has an inner surface and a fastening material that is disposed on the inner surface. A strap extends outwardly from the outer shell. The strap has an attachment end that is selectively coupled to the outer shell. A fastener is operably coupled to the attachment end of the strap. The fastener includes a first coupling feature that is selectively coupled to at least one of the outer shell and the strap. An inner liner includes a first layer and a second layer, and the first layer is operably coupled to the inner surface of the outer shell. Cannabidiol oil is disposed on the second layer of the inner layer.

According to another aspect, a medication that includes a cannabidiol oil and is disposed on the second layer of the inner layer.

According to another aspect, a first layer of an inner liner includes a phase change material.

According to another aspect, a protective sleeve assembly further includes a carrier oil, wherein a cannabidiol oil and the carrier oil are disposed on a second layer of an inner liner.

According to another aspect, a carrier oil is a medium chain triglyceride oil.

According to another aspect, an inner liner includes a second fastening material that is selectively and removable coupled to a fastening material of an outer shell, wherein the inner liner is configured to be repeatedly removed from an outer shell and replaced.

According to another aspect, a protective sleeve assembly further includes a bounded end defined by an outer shell and an inner liner and is configured to provide padding for a digit within said protective sleeve.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or moveable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure as shown in the exemplary embodiments is illustrative only. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A protective sleeve for a digit, comprising:
an outer shell defining slits and having a tapered configuration;
straps extending outwardly from and operably coupled to the outer shell, the straps each including an attachment end and a stabilizing portion coupled to the outer shell;
a fastener operably coupled to the attachment end and selectively coupled to at least one of the outer shell and the stabilizing portions of the straps;
an inner liner operably coupled to the outer shell to define a pocket between the inner liner and the outer shell, the inner liner including a plurality of layers and comprising an absorbent material;
a rigid member disposed within the pocket via one of the slits defined by the outer shell; and
an oil disposed on at least one layer of the plurality of layers of the inner liner.

2. The protective sleeve of claim 1, wherein the oil is comprised of a cannabidiol oil.

3. The protective sleeve of claim 2, wherein the oil is comprised of the cannabidiol oil and a carrier oil.

4. The protective sleeve of claim 1, wherein the straps further include an elastic portion disposed between the attachment end and the stabilizing portion.

5. The protective sleeve of claim 1, wherein at least one layer of the plurality of layers includes a medication layer, and wherein the absorbent material is disposed over the medication layer.

6. The protective sleeve of claim 1, wherein at least one layer of the plurality of layers comprises a phase change material.

7. A digit protection assembly, comprising:
an outer shell comprising a moisture repellant material;
a strap operably coupled to the outer shell, the strap including a fastener operably coupled to an attachment end of the strap;
an inner liner operably coupled to the outer shell and defining a pocket between the inner liner and the outer shell, the inner liner comprising an absorbent material; and
a medication disposed on the inner liner, the medication comprising a cannabidiol oil.

8. The digit protection assembly of claim 7, wherein the cannabidiol oil comprises 20% of the medication.

9. The digit protection assembly of claim 7, wherein the inner liner includes a first layer and a second layer, and wherein the first layer includes a phase change material.

10. The digit protection assembly of claim 9, wherein the second layer includes the absorbent material and the medication.

11. The digit protection assembly of claim 7, wherein the outer shell has an inner surface and a fastening material disposed on the inner surface, and wherein the inner liner includes a second fastening material reciprocally coupled to the fastening material of the outer shell.

12. The digit protection assembly of claim 7, wherein the outer shell defines a slit along a lower edge of the outer shell.

13. The digit protection assembly of claim 12, further comprising:
a rigid member extending through the slit defined by the outer shell and disposed within the pocket defined between the inner liner and the outer shell.

14. A protective sleeve assembly, comprising:
an outer shell having an inner surface and a fastening material disposed on the inner surface;
a strap extending outwardly from the outer shell, the strap having an attachment end selectively coupled to the outer shell;
a fastener operably coupled to the attachment end of the strap, the fastener including a first coupling feature selectively coupled to at least one of the outer shell and the strap;
an inner liner including a first layer and a second layer, wherein the first layer is operably coupled to the inner surface of the outer shell; and
a cannabidiol oil disposed on the second layer of the inner liner.

15. The protective sleeve assembly of claim 14, further comprising:
a medication including the cannabidiol oil and is disposed on the second layer of the inner liner.

16. The protective sleeve assembly of claim 14, wherein the first layer of the inner liner includes a phase change material.

17. The protective sleeve assembly of claim 14, further comprising:
a carrier oil, wherein the cannabidiol oil and the carrier oil are disposed on the second layer of the inner liner.

18. The protective sleeve assembly of claim 17, wherein the carrier oil is a medium chain triglyceride oil.

19. The protective sleeve assembly of claim 14, wherein the inner liner includes a second fastening material selectively and removably coupled to the fastening material of the outer shell, and wherein the inner liner is configured to be repeatedly removed from the outer shell and replaced.

20. The protective sleeve assembly of claim 14, further comprising:
a bounded end defined by the outer shell and the inner liner and configured to provide padding for a digit within said protective sleeve.
